(19) United States
(12) Patent Application Publication
(10) Pub. No.: US 2023/0113721 A1
(43) Pub. Date: Apr. 13, 2023

(54) FUNCTIONAL MEASUREMENTS OF VESSELS USING A TEMPORAL FEATURE

(71) Applicant: MEDHUB LTD, Tel Aviv (IL)

(72) Inventors: ALEXANDRE KASSEL, TEL AVIV (IL); NATANEL DAVIDOVITS, TEL AVIV (IL); OR BRUCH-EL, TEL AVIV (IL)

(21) Appl. No.: 17/914,341

(22) PCT Filed: Mar. 25, 2021
(86) PCT No.: PCT/IL2021/050341

§ 371 Date: Sep. 25, 2022

Related U.S. Application Data
(60) Provisional application No. 62/994,895, filed on Mar. 26, 2020.

(51) Int. Cl. G06T 7/00; G06T 7/73; G06T 7/246; G06T 7/90; G16H 30/40; G16H 70/60

(52) U.S. Cl. CPC G06T 7/0016; G06T 7/73; G06T 7/90; G06T 7/248; G16H 30/40; G16H 70/60; G06T 2207/10016; G06T 2207/10116; G06T 2207/30104

(57) ABSTRACT

Embodiments of the invention provide a system and method for determining an FFR value for a pathology in a vessel. A value of a predetermined attribute is extracted from a location of the pathology in an image of the vessel and a temporal feature is calculated based on the value of the predetermined attribute. The temporal feature is input to an estimator and an FFR value for the pathology is obtained from an output of the estimator. A structural feature of the pathology may also be input to the estimator to obtain the FFR value based on the temporal feature and the structural feature.

### FIELD

The present invention relates to automated vessel (e.g., blood vessels) analysis from image data and automatic determination of functional measurements, such as fractional flow reserve (FFR).

### BACKGROUND

Artery diseases involve circulatory problems in which pathologies in arteries may cause reduced blood flow to body organs.

Angiography is an X-ray technique used in the examination of arteries, veins and organs to diagnose and treat blockages and other blood vessel problems. During an angiogram, a catheter is inserted into an artery or vein from an access point and a contrast agent is injected through the catheter to make the blood vessels visible on the angiogram X-ray image.

Fractional flow reserve (FFR) is a technique used to measure pressure differences across a stenosis (narrowing of a vessel, usually due to atherosclerosis) to determine the likelihood that the stenosis impedes oxygen delivery to the heart muscle. FFR is defined as the pressure after (distal to) a stenosis relative to the pressure before the stenosis. The result is an absolute number; for example, an FFR of 0.80 means that a given stenosis causes a 20% drop in blood pressure. In other words, FFR expresses the maximal flow down a vessel in the presence of a stenosis compared to the maximal flow in the hypothetical absence of the stenosis. FFR is typically measured during maximal blood flow (hyperemia).

Some methods use calculations of speed and dynamics of blood flow through vessels (e.g., by using computational fluid dynamics (CFD)), as a less invasive alternative to conventional FFR measurement. However, performing simulation of flow in a coronary vessel requires a 3D description of the vessel lumen. A 3D description of a vessel can be achieved by employing specific imaging modalities, (such as a multi-slice computed tomography (CT) scanner, an angiography unit capable of rotational coronary angiography, 3D quantitative coronary angiography (3D-QCA), etc.) or by reconstructing a full 3D model from images, which is a typically slow process that requires heavy use of immediately available memory.

### SUMMARY

Embodiments of the invention enable automatically determining FFR and/or other functional measurements of vessels, from an X-ray angiogram, without having to reconstruct a 3D model of the vessels.

Systems and methods according to embodiments of the invention, use temporal features extracted from a sequence of images of the vessels, for improved accuracy of functional measurements at a pathology site (e.g., stenosis) in a vessel.

In some embodiments, a typically visible attribute at a location of a pathology in an image of a vessel is recorded over time, in consecutive images, to create a signal. Temporal features are extracted from the signal and may be input to a machine learning estimator to provide a functional measurement value relevant to the location of the pathology.

There is provided, in accordance with an embodiment of the invention, a system for analysis of a vessel, which includes a processor configured to receive a plurality of images of a patient’s vessel (e.g., an angiogram video) and determine the location of a pathology in the vessel, in at least some of the plurality of images. The processor can then create a signal describing a predetermined attribute at the location of the pathology, over time, and determine a value of a functional measurement for the pathology, based on the signal. The functional measurement may then be displayed on a user interface device.

In some embodiments a temporal feature is extracted from the signal and the value of the functional measurement is determined based on the temporal feature. The temporal feature may be input to an estimator, possibly together with a structural feature of the pathology, to determine the value of the functional measurement.

In some embodiments the temporal feature includes a calculation of a combination of attribute values determined from the plurality of images.

In some embodiments the processor assigns a weight to each attribute determined from the plurality of images, to obtain weighted attribute values, and calculates a combination of the weighted attribute values to create the signal.

The processor may determine the location of the pathology based on structural features of the vessel in at least one image from the plurality of images. In some embodiments the processor may determine the location of the pathology in a first image from the plurality of images, track the pathology in subsequent images from the plurality of images to determine the location of the pathology in the subsequent images, and determine a value of the predetermined attribute at each location in each of the subsequent images, to create the signal.

In another aspect of the invention there is provided a method for determining an FFR value for a pathology in a vessel. The method includes the steps of extracting, from a location of a pathology in images of the vessel, values of a predetermined attribute, calculating a temporal feature based on the values of the predetermined attribute, inputting the temporal feature to an estimator, and obtaining, from an output of the estimator, an FFR value for the pathology. In some embodiments the method includes displaying the FFR value on a user interface device.

A structural feature of the pathology may also be input to the estimator to obtain the FFR value based on the temporal feature and the structural feature.

In some embodiments the method includes tracking the location of the pathology throughout a plurality of images of the vessel and extracting a value of the predetermined attribute from the location of the pathology in at least some of the plurality of images. The temporal feature may include a calculation of the values of the predetermined attributes extracted from the plurality of images. Possibly, the temporal feature includes a calculation of weighted attributes.

In some embodiments the method includes assigning a weight to each of the predetermined attributes extracted from the plurality of images, based on a probability of pathology detection in each image of the plurality of images.

In some embodiments, an FFR value (or other functional measurement) for a pathology may be calculated based on a combination of attributes extracted from images of the vessel captured from different angles and/or based on a combination of functional measurement values obtained for the pathology from images capturing the pathology from different angles.

Thus, in one embodiment the method includes extracting a first value of the predetermined attribute from the location of the pathology in an image captured from a first angle and extracting a second value of the predetermined attribute from the location of the pathology in an image captured from a second angle. The first and second values of attribute are combined to obtain the FFR value for the pathology.

In another embodiment the method includes obtaining a first FFR value of the pathology in an image of the vessel captured from a first angle and obtaining a second FFR value of the pathology in an image of the vessel captured from a second angle. The first and second values of FFR may be combined to obtain a more accurate FFR value for the pathology.

### BRIEF DESCRIPTION OF THE FIGURES

The invention will now be described in relation to certain examples and embodiments with reference to the following illustrative figures so that it may be more fully understood. In the drawings:

FIG. 1 schematically illustrates a system for determining a functional measurement of a vessel, according to embodiments of the invention;

FIG. 2 schematically illustrates a method for determining a functional measurement of a vessel from a video, according to embodiments of the invention;

FIGS. 3A and 3B schematically illustrate a method for creating a signal based on attributes extracted from a plurality of images, according to embodiments of the invention; and

FIG. 4 schematically illustrates a system and method for determining a functional measurement of a vessel using a temporal feature, according to embodiments of the invention.

### DETAILED DESCRIPTION

Embodiments of the invention provide methods and systems for determining a value of a functional measurement (such as FFR) at a location of a pathology, such as a stenosis, in a vessel, by using temporal features extracted from images of a video of the vessel.

A “vessel” may include a tube or canal in which body fluid is contained and conveyed or circulated. Thus, the term vessel may include blood veins or arteries, coronary blood vessels, lymphatics, portions of the gastrointestinal tract, etc.

An image of a vessel may be obtained using suitable imaging techniques, for example, X-ray imaging, ultrasound imaging, Magnetic Resonance imaging (MRI) and others suitable imaging techniques.

A pathology may include, for example, a narrowing of the vessel (e.g., stenosis or stricture), lesions within the vessel, etc.

A “functional measurement” is a measurement of the effect of a pathology on flow through the vessel. Functional measurements may include measurements such as an estimate of FFR, an estimate of instant flow reserve (iFR), coronary flow reserve (CFR), quantitative flow ratio (QFR), resting full-cycle ratio (RFR), quantitative coronary analysis (QCA), and more.

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well known features may be omitted or simplified in order not to obscure the present invention.

Unless specifically stated otherwise, as apparent from the following discussions, it is appreciated that throughout the specification discussions utilizing terms such as “using”, “analyzing”, “processing,” “computing,” “calculating,” “determining,” “detecting”, “identifying”, “choosing”, “producing”, “providing”, “extracting” or the like, refer to the action and/or processes of a computer or computing system, or similar electronic computing device, that manipulates and/or transforms data represented as physical, such as electronic, quantities within the computing system’s registers and/or memories into other data similarly represented as physical quantities within the computing system’s memories, registers or other such information storage, transmission or display devices. Unless otherwise stated, these terms refer to automatic action of a processor, independent of and without any actions of a human operator.

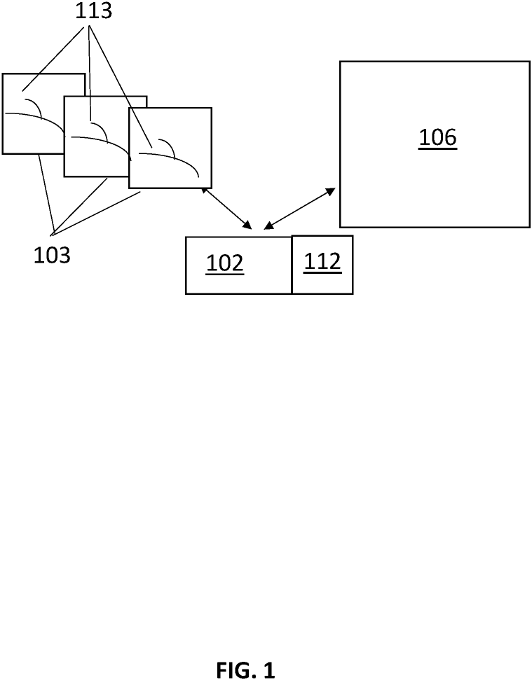

In one embodiment, which is schematically illustrated in FIG. 1, a system for analysis of a vessel includes a processor 102, which may be in communication with a user interface device 106. Processor 102 receives one or more images 103 of a vessel 113. In the embodiments exemplified herein the images 103 are X-ray 2D lengthwise images of a patient’s vessels. Images 103 may be consecutive images, typically forming a video (e.g., a video angiogram) that can be displayed via the user interface device 106. In other embodiments, images 103 include images of a vessel 113 captured from different viewpoints or angles.

Processor 102 performs analysis on the received image(s) and may communicate analysis results and/or instructions or other communications, based on the analysis results, to a user, e.g., via the user interface device 106. In some embodiments, user input can be received at processor 102, via user interface device 106.

Vessels 113 may include one or more vessel or portion of a vessel, such as a vein or artery, a branching system of arteries (arterial trees) or other portions and configurations of vessels.

Processor 102 may include, for example, one or more processors and may be a central processing unit (CPU), a graphics processing unit (GPU), a digital signal processor (DSP), a field-programmable gate array (FPGA), a microprocessor, a controller, a chip, a microchip, an integrated circuit (IC), or any other suitable multi-purpose or specific processor or controller. Processor 102 may be locally embedded or remote, e.g., on the cloud.

Processor 102 is typically in communication with a memory unit 112. In one embodiment the memory unit 112 stores executable instructions that, when executed by the processor 102, facilitate performance of operations of the processor 102, as described below. Memory unit 112 may also store image data (which may include data such as pixel values that represent the intensity of light having passed through body tissue and received at an imaging sensor, as well partial or full images or videos) of at least part of the images 103.

Memory unit 112 may include, for example, a random access memory (RAM), a dynamic RAM (DRAM), a flash memory, a volatile memory, a non-volatile memory, a cache memory, a buffer, a short term memory unit, a long term memory unit, or other suitable memory units or storage units.

The user interface device 106 may include a display, such as a monitor or screen, for displaying images, instructions and/or notifications to a user (e.g., via graphics, images, text or other content displayed on the monitor). User interface device 106 may also be designed to receive input from a user. For example, user interface device 106 may include or may be in communication with a mechanism for inputting data, such as a keyboard and/or mouse and/or touch screen, to enable a user to input data, instructions and other inputs.

All or some of the components of the system may be in wired or wireless communication, and may include suitable ports such as USB connectors and/or network hubs.

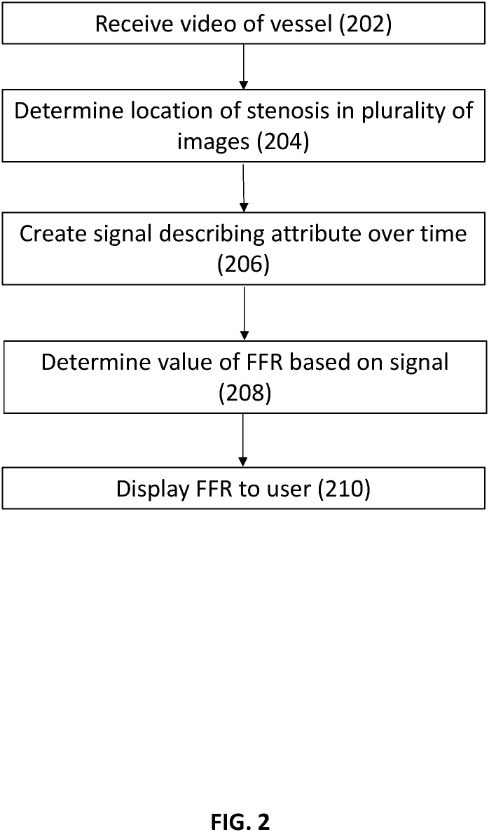

In one embodiment, which is schematically illustrated in FIG. 2, processor 102 receives a video of a vessel (step 202), e.g., an angiogram video of a patient’s arteries, and determines the location of a pathology such as a stenosis, in a plurality of images of the video (step 204). Processor 102 then detects one or more predetermined attribute in the images, at the location of the pathology, and creates a signal describing the attribute(s) over time (step 206). In some embodiments, a combination of attributes is used to create the signal. For example, an average (or other statistic) of several attribute values may be used to create a signal or temporal feature. In some embodiments, attributes may be assigned weights prior to being combined. For example, a weight may be assigned to an attribute extracted from a specific image based on the level of confidence or probability of pathology detection in that image. Thus, for example, an attribute extracted from an image in which a pathology was detected at a higher probability will be assigned a higher weight than an attribute extracted from an image in which a pathology was detected at a lower probability. The weighted attributes may then be combined to provide, for example, a weighted average (or other statistic) from which a temporal feature may be calculated.

The predetermined attribute may be a visible characteristic of the images or other characteristic inherent to the image. For example, the predetermined attribute may include pixel intensity or pixel color or grey levels. In one example, the attribute may include the number of pixels having a color or intensity above a threshold and/or the distribution of these pixels.

Processor 102 then determines a value of a functional measurement (e.g., FFR value) for the pathology, based on the signal (step 208).

Processor 102 may cause the value of the functional measurement and/or an indication of the value to be displayed to a user (step 210), e.g., on user interface device 106.

In some embodiments processor 102 may also cause an indication of the pathology to be displayed, via the user interface device 106, alone or together with the indication of the value of the functional measurement. An indication of a pathology displayed on a display of a user interface device may include, for example, graphics, such as, letters, numerals, symbols, different colors and shapes, etc., that can be superimposed on the image or video of the patient’s vessels.

In one embodiment processor 102 determines the location of a pathology (in step 204) by using computer vision techniques, without requiring user input regarding a location of the vessels in the image and/or location of the pathology. For example, processor 102 may apply a segmenting algorithm on at least one image from the video, to obtain an image of segmented out vessels, also referred to as a vessels mask. Processor 102 may then apply a classifier on the image to obtain, from output of the classifier, an indication of a presence of a pathology (e.g., stenosis) in the vessels and a location of the pathology. Classifiers, such as DenseNet, CNN (Convolutional Neural Network) or EfficientNet, may be used.

The location of the pathology may be an x,y location on a coordinate system describing the image and/or an anatomical location, e.g., based on description of the location relative to structures of the vessel. For example, a segmenting algorithm can be used to determine which pixels in the image are part of the pathology and the location of the pathology relative to structures of the vessel can be recorded, e.g., in a lookup table or other type of virtual index. In some embodiments processor 102 creates a virtual mark which is specific per pathology, and in a case of multiple pathologies in a single image, distinguishes the multiple pathologies from one another. The pathology can then be detected in subsequent images of the vessel, based on the virtual mark.

In some embodiments, processor 102 obtains a vessels mask by using semantic segmentation algorithms on the image. A machine learning model can be used for the segmentation, e.g., deep learning models such as Unet or FastFCN or other deep learning based semantic segmentation techniques.

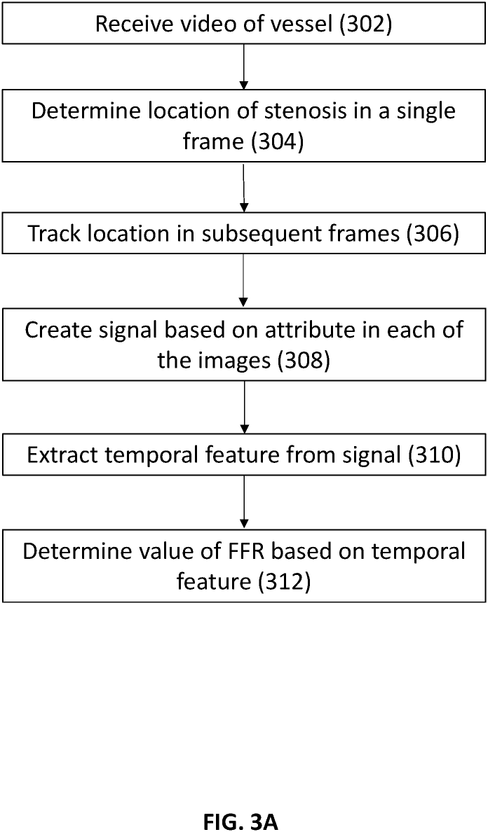

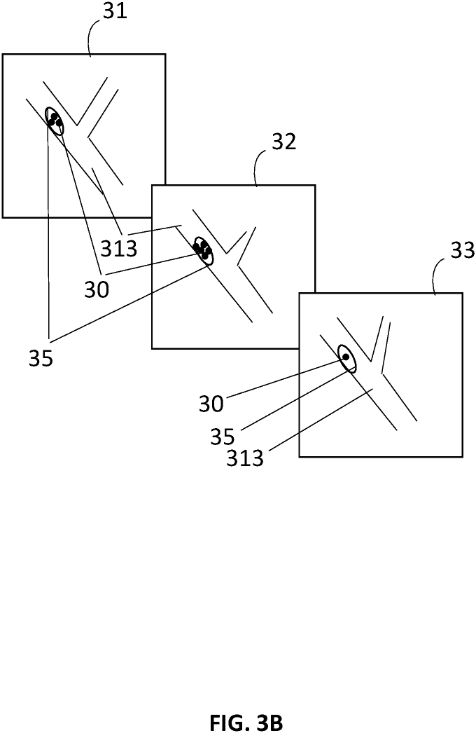

In one embodiment, which is schematically illustrated in FIG. 3A, the location of a pathology is determined in a first image of a video and is then tracked in subsequent images of the video to determine the location of the pathology in each of the subsequent images. Thus, processor 102 receives a video of a vessel, such as a patient’s arteries (step 302) and determines the location of a pathology, such as a stenosis, in a single image or frame (step 304). The first image may be an optimal image, selected from a plurality of images of the patient’s vessels, as the image showing the most detail. In the case of angiogram images, which include contrast agent injected to a patient to make vessels (e.g., blood vessels) visible on an X-ray image, an optimal image may be an image of a blood vessel showing a large/maximum amount of contrast agent. An optimal image can be detected by applying image analysis algorithms on images or frames of the video, e.g., to detect a maximal amount of contrast agent.

In one embodiment an image captured at a time corresponding with maximum heart relaxation is an image showing a maximum amount of contrast agent. Thus, an optimal image may be detected based on capture time of the images compared with, for example, measurements of electrical activity of the heartbeat (e.g., ECG printout) of the patient.

The location of the pathology which was detected in step 304 is then tracked in subsequent images of the video (step 306) to determine the location of the pathology in each of the subsequent images. The location of the pathology may be tracked, for example, by applying optical flow techniques on the images and marking the pathology (e.g., as described above). This enables detecting the same pathology in each of the images, even if it’s shape or other visual characteristics change in between images of the video.

Since the location of the pathology can be determined in each image of the video, processor 102 can determine a value of one or more predetermined attribute at each location in each of the subsequent images. Processor 102 can create a signal based on the attribute(s) in each of the images (step 308) and can extract a temporal feature from the signal (step 310). Processor 102 may then determine the FFR value based on the temporal feature (step 312).

A temporal feature may include a calculation of attribute values in a plurality of images (e.g., images of a video). For example, a temporal feature may include a statistic (e.g., average or mean) of attribute values and/or another calculation, such as an addition, subtraction, multiplication and/or division of attribute values. A temporal feature may also include calculations of a graphic or other representation of attribute values.

FIG. 3B schematically illustrates an example of a predetermined attribute and a signal created from the attribute in images 31, 32 and 33, which are subsequent images in a video, such as an X-ray angiogram video. Due to the pumping action of the heart, there is some movement of vessels 313 of a patient so that visual characteristics of the vessels 313 and/or of pathologies (e.g., pathology 35) may change slightly in consecutive images of a video. Thus, an attribute in the images 31, 32 and 33 may have different values in each image.

One or a combination of attributes may be used. Attributes may include a visible characteristic of the images or other characteristics inherent to the image, for example, the intensity of the image at the area of the pathology.

In the example illustrated in FIG. 3B, the attribute includes the number of pixels having a grey level above a threshold. Processor 102 detects the pixels 30 having a grey level above the threshold, at the location of a pathology 35 in vessel 313. Processor 102 then creates a signal describing the different numbers of pixels 30 in each of images 31, 32 and 33. A temporal feature, such as the difference between a maximum and minimum number of pixels (e.g., the number of pixels 30 in image 32 and image 33, correspondingly) and/or the slope of a graph describing the different numbers of pixels versus time, may be extracted from the signal and used for determining a functional measurement associated with the location of pathology 35. Other attributes and/or temporal features may be used for determining a functional measurement associated with the location of pathology 35, according to embodiments of the invention.

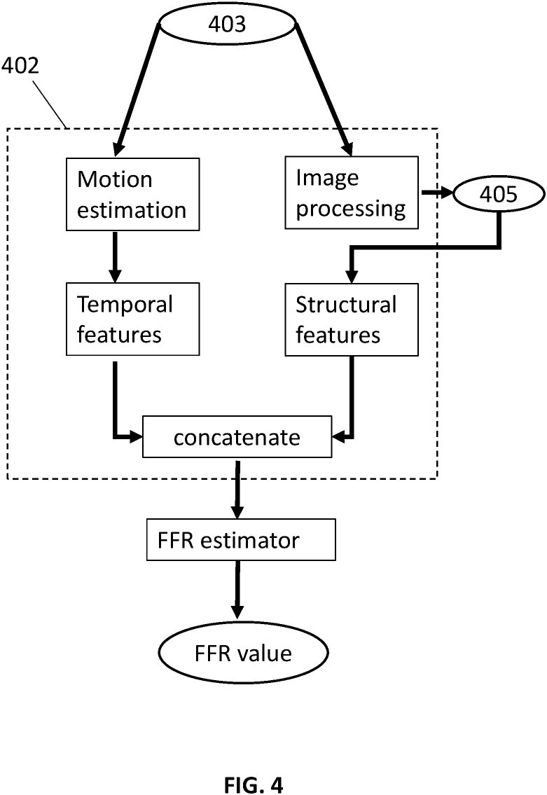

For example, as schematically illustrated in FIG. 4, a sequence of angiogram X-ray images 403 (e.g., one or more angiogram X-ray video and/or images captured from different angles) of a patient are processed by processor 402. In some embodiments, processor 402 applies image processing techniques on the images 403 to choose an optimal image 405 (e.g., an image showing a large amount of contrast agent, which is indicative of hyperemia). The optimal image 405 is then processed to extract structural features of a pathology. For example, the optimal image 405 may be segmented to extract a binary map of the vessels. A centerline of the vessels may be determined and the distance between the centerline and the borders of the vessels may be extracted. This distance and/or other such calculations may produce structural features. A location of a pathology in optimal image 405 may be determined based on the structural features.

In parallel, motion estimation is performed on the angiogram X-ray video. The motion estimation determines optical flow to estimate motion of blood (as represented by the contrast agent) within the imaged arteries/veins. Based on the motion estimation the location of the pathology detected in optimal image 405, can be tracked throughout images 403.

Processor 402 extracts one or more temporal features (e.g., as described above) related to the location of the pathology, based on the tracking.

The structural features and temporal features are then concatenated and input to an FFR estimator, which may include a machine learning component, to predict an FFR value at the location of the pathology.

In one embodiment the FFR estimator includes a machine learning model running a regression algorithm. For example, the machine learning algorithm can be implemented by using the XGBoost algorithm or other Gradient Boosted Machine or decision trees regression. In other examples, neural network or deep neural network based regression can be used. Other learning algorithms that are not regressors may also be used.

In some embodiments, a value of a functional measurement (e.g., FFR value) for a pathology may be calculated based on a combination of attributes extracted from images of the vessel captured from different angles and/or based on a combination of functional measurement values obtained for the pathology from images capturing the pathology from different angles.

In one embodiment, values of a predetermined attribute from the location of a pathology in images captured from different angles, are combined to obtain the FFR value for the pathology, typically the FFR value that will be displayed to a user. For example, an average, weighted average (e.g., as described herein) or other statistic may be calculated from the different values of attribute. The calculated average (or other statistic) may then be input to the estimator, or otherwise used, to provide an FFR value for the pathology.

In another embodiment different FFR values of the pathology may be obtained (e.g., as described herein) from images of the vessel captured from different angles. The different FFR values may then be combined, as described above to provide a single FFR value for the pathology, typically the value that will be displayed to the user.

The use of FFR values obtained from images capturing the vessel from different angles and the use of temporal features extracted from a plurality of images of the vessel, facilitates operation of the FFR estimator and provides more accurate functional measurements for a pathology site (e.g., stenosis) in a vessel.

1. A system for analysis of a vessel, the system comprising a processor to:
receive a plurality of images of a patient’s vessel;
determine location of a pathology in the vessel, in at least some of the plurality of images;
create a signal describing a predetermined attribute at the location of the pathology, over time;
determine a value of a functional measurement for the pathology, based on the signal; and
display the value on a user interface device.

2. The system of claim 1 wherein the processor is to determine the location of the pathology based on structural features of the vessel in at least one image from the plurality of images.

3. The system of claim 1 wherein the processor is to:
determine the location of the pathology in a first image from the plurality of images;
track the pathology in subsequent images from the plurality of images to determine the location of the pathology in the subsequent images; and
determine a value of the predetermined attribute at each location in each of the subsequent images, to create the signal.

4. The system of claim 3 wherein the first image shows a maximum amount of contrast agent.

5. The system of claim 1 wherein the processor is to extract a temporal feature from the signal and determine the value of the functional measurement based on the temporal feature.

6. The system of claim 5 wherein the processor is to input the temporal feature to an estimator to determine the value of the functional measurement.

7. The system of claim 6 wherein the processor is to input a structural feature of the pathology to the estimator, to determine the value of the functional measurement.

8. The system of claim 6 wherein the estimator comprises a regressor.

9. The system of claim 5 wherein the temporal feature comprises a calculation of a combination of attribute values determined from at least some of the plurality of images.

10. The system of claim 9 wherein the processor is to:
assign a weight to each attribute determined from the plurality of images, to obtain weighted attribute values; and
calculate a combination of the weighted attribute values to create the signal.

11. The system of claim 1 wherein the predetermined attribute comprises pixel intensity or pixel color or grey level.

12. The system of claim 1 wherein the predetermined attribute comprises an amount of pixels having a color or grey level above a threshold.

13. A method for determining an FFR value for a pathology in a vessel, the method comprising:
extracting, from a location of the pathology in a plurality images of the vessel, values of a predetermined attribute;
calculating a temporal feature based on the values of the predetermined attribute;
inputting the temporal feature to an estimator; and
obtaining, from an output of the estimator, an FFR value for the pathology.

14. The method of claim 13 comprising inputting a structural feature of the pathology to the estimator to obtain the FFR value based on the temporal feature and the structural feature.

15. The method of claim 13 comprising:
tracking the location of the pathology throughout the plurality of images of the vessel; and
extracting a value of the predetermined attribute from the location of the pathology in at least some of the plurality of images,
wherein the temporal feature comprises a calculation of the values of the predetermined attributes extracted from the plurality of images.

16. The method of claim 15 wherein the temporal feature comprises a calculation of weighted attributes.

17. The method of claim 16 comprising assigning a weight to each of the predetermined attributes extracted from the plurality of images, based on a probability of pathology detection in each image of the plurality of images.

18. The method of claim 13 comprising
extracting a first value of the predetermined attribute from the location of the pathology in an image captured from a first angle;
extracting a second value of the predetermined attribute from the location of the pathology in an image captured from a second angle; and
combining the first and second values of attribute to obtain the FFR value for the pathology.

19. The method of claim 13 comprising
obtaining a first FFR value of the pathology in an image of the vessel captured from a first angle;
obtaining a second FFR value of the pathology in an image of the vessel captured from a second angle; and
combining the first and second values of FFR to obtain the FFR value for the pathology.

20. The method of claim 13 comprising displaying the FFR value for the pathology on a user interface device.
